(19) United States
(12) Patent Application Publication
(10) Pub. No.: US 2026/0174257 A1
(43) Pub. Date: Jun. 25, 2026

(54) BEDDING CAPABLE OF DETECTING SLEEPING POSTURE AND METHOD THEREOF

(71) Applicant: XIAMEN BELUGA TECHNOLOGY CO., LTD., Xiamen (CN)

(72) Inventors: Chenxu Lu, Xiamen (CN); Zhenlei Liu, Xiamen (CN); Tongzhi Li, Xiamen (CN); Zehai Chen, Xiamen (CN); Dianting Zeng, Xiamen (CN); Ying Lin, Xiamen (CN); Long Cheng, Xiamen (CN); Jinhuang Kang, Xiamen (CN); Hua Yuan, Xiamen (CN); Jiangping Miao, Xiamen (CN); Jianhe Zheng, Xianmen (CN); Xuan Zheng, Xiamen (CN)

(21) Appl. No.: 19/407,760

(22) Filed: Dec. 3, 2025

(30) Foreign Application Priority Data
Dec. 4, 2024 (CN) 202411773179.4

(51) Int. Cl. A47G 9/10; A47C 20/04; A61B 5/00; A61B 5/11

(52) U.S. Cl. CPC A47G 9/109; A47G 9/1027; A61B 5/1116; A61B 5/4806; A61B 5/4836; A61B 5/6892; A47C 20/048; A61B 2562/0247; A61B 2562/043

(57) ABSTRACT

The present disclosure relates to a bedding capable of detecting sleeping posture and a method therefor, comprising: a main body portion (1), in which an airbag (5) is internally provided, an outer shape of the main body portion (1) having a front side end surface for a shoulder of a user in sleep to approach; a shoulder sensor group, comprising a plurality of shoulder sensors (2/3) arranged in an array along a long side direction of the front side end surface; and a controller (6) connected to the airbag (5) and the shoulder sensor group, configured to determine a sleeping posture of the user according to a detection result of the shoulder sensor group, and further regulate inflation and deflation of the airbag to change a height of the main body portion (1), wherein, when viewed from a side along the long side direction of the front side end surface, the front side end surface has a downwardly and inwardly recessed shape, and the shoulder sensor group is positioned at a downwardly and inwardly recessed position of the front side end surface, such that the shoulder of the user during side sleeping is able to be close to the shoulder sensor group.

### PRIORITY DATA

This application claims the benefit of Chinese patent application serial No. 202411773179.4, filed Dec. 4, 2024, which is hereby incorporated by reference in its entirety.

### TECHNICAL FIELD

The present disclosure relates to the field of smart homes. More specifically, the present disclosure relates to a bedding capable of detecting sleeping posture and a method thereof.

### BACKGROUND

In recent years, in the field of health and wellness, it is well known that sleep has become a key factor affecting health, and people's emphasis on sleep has increased year by year. As a smart home solution, accompanied by the rapid intelligentization of bedding such as pillows and mattresses involved in the sleep industry, requirements for helping to eliminate sleep disorders caused by various factors and improving the sleep quality of sub-healthy populations are increasingly high, whether in specific professional institutions such as medical care and elderly care or in the field of daily consumption by ordinary residents. Consequently, a variety of smart bedding such as smart pillows and smart mattresses have emerged.

From the perspective of ergonomics, the physiological curves of the human body are different when lying on the back (supine) and lying on the side. Regarding the height of bedding such as pillows, according to statistical laws, it is roughly based on the shoulder-zygomatic distance when lying on the side, and based on the neck-back distance when lying on the back. Especially for adults, there is often a large gap between the shoulder-zygomatic distance and the neck-back distance. In order to maintain the natural curvature of the spine, a lower pillow is required when lying on the back, while a higher pillow is required when lying on the side. Therefore, a pillow with a fixed height cannot be suitable for both sleeping postures simultaneously. In response to this, partition pillow designs have been proposed in the prior art, adapting to the needs of different sleeping postures by setting a higher side sleeping area and a lower back sleeping area.

The human body is in a quasi-static state during sleep, and maintaining a specific posture statically for a long time will lead to poor local blood circulation in the human body, and the brain will immediately issue a turning command to improve discomfort. Medical research has found that normal people adjust their sleeping posture on average about every half hour during sleep. In the use of the above-mentioned partition pillows, since people unconsciously turn over frequently during sleep, it is difficult to always correspond to the correct partition. Therefore, in practice, a smart pillow capable of automatically adjusting its height according to the person's sleeping posture has become a better solution. Therefore, in order to realize the adaptive adjustment function as the core function of smart bedding, real-time and accurate detection of various sleeping postures including side (lateral) sleeping, back (supine) sleeping, etc., is of great significance for the automatic shape adjustment of smart bedding and various automatic intervention functions including snoring cessation.

In existing sleeping posture detection technologies, methods for detection external to the bedding include, for example: sensing body posture through wearable devices such as three-axis accelerometers; image recognition through cameras; analyzing pressure distribution through pressure sensor arrays arranged on the mattress; or detecting carbon dioxide concentration, body temperature, or infrared signals. However, the above-mentioned external detection technologies have defects. For example, accelerometers need to be worn on the body, resulting in poor user experience; camera shooting and image recognition raise concerns about personal privacy; arranging pressure sensor arrays on the mattress leads to increased equipment and limited scope of use; and detection based on physiological indicators often requires real-time monitoring, requires high equipment standards, leads to high costs, and has unstable detection accuracy. Therefore, in practice, it is more preferable to implement sleeping posture detection inside the body of bedding such as pillows.

As a way to implement sleeping posture detection inside bedding, the current mainstream technology is based on pressure detection. Detection sites include, for example, the head and neck of the human body, the rear of the shoulders and the upper back when lying on the back, and the side of the upper arm and the acromion area when lying on the side. The types of sensors used include, for example: switch-type sensors, which make determination by detecting the presence or absence of pressure; piezoresistive sensors, where the greater the pressure, the smaller the resistance and the larger the measured value; piezoelectric sensors, which are sensitive to pressure changes and can be used to collect physiological information such as heartbeat micro-shock signals; air pressure sensors, which indirectly detect pressure by measuring air pressure changes in an airbag; and capacitive sensors, including two-sheet and single-sheet types, wherein the two-sheet type constitutes a capacitor through a compressible insulating layer between positive and negative electrode sheets (the greater the pressure, the larger the capacitance), and the single-sheet type constitutes a capacitor based on the fit between the sensor and the human body (the closer the fit, the larger the capacitance).

Regarding the types of detection data used as the basis for sleeping posture determination, according to the detection principle, they can be roughly divided into, for example, pressure area, total pressure magnitude, and pressure spatial distribution. In the case of using pressure area data, determination is mainly made indirectly based on the difference in pressure caused by the difference in the compressed area when lying on the back and lying on the side, such as by the time for inflating a detection airbag or the number of switch-type sensors turned on due to compression. However, there are problems such as affecting sleep experience, poor real-time performance, and low accuracy. In the case of using total pressure magnitude data, direct determination is mainly made based on the difference in downward pressure of the head when lying on the back and lying on the side. However, the downward pressure of the head is easily affected by changes in the relative positional relationship between the human body and the bedding, the height of the bedding itself, interference from internal components, and even bedding supplies including pillowcases, easily leading to low accuracy.

On the other hand, in the case of using pressure spatial distribution data for sleeping posture detection, determination is mainly made based on the difference in the spatial distribution of pressure on the shoulders and back when lying on the back and lying on the side, which can avoid the drawbacks of the above two situations and thus becomes a more accurate and reliable data type in actual usage scenarios. In the prior art, there is a solution of measuring shoulder pressure through a strip-shaped piezoresistive sensor extending from the side of the pillow or other bedding close to the human shoulder. However, the configuration of the strip-shaped sensor will cause a foreign body sensation for the user, especially when lying on the side. The shoulder is blocked by the sensor and cannot fit comfortably to the lower side position of the pillow, thereby affecting the sleep experience; in addition, due to long-term squeezing by the user's shoulder, the life of the sensor will be affected.

In view of this, the industry urgently needs an improved bedding with a mechanism for sleeping posture detection and adaptive adjustment that can simultaneously achieve high sleeping posture detection accuracy, low cost, and user-friendliness.

### SUMMARY

In response to the problems of the above-mentioned existing sleeping posture detection technologies, inventors of the present application have conducted in-depth research and development and proposed improved sleeping posture detection and adaptive adjustment solutions, including bedding and methods capable of performing sleeping posture detection based on shoulder pressure.

A brief summary regarding the present disclosure is given below to provide a basic understanding of some aspects of the present disclosure. However, it should be understood that this summary is not an exhaustive overview of the present disclosure. It is not intended to identify key or important parts of the present disclosure, nor is it intended to limit the scope of the present disclosure. Its purpose is merely to present some concepts regarding the present disclosure in a simplified form as a prelude to the more detailed description given later.

According to one aspect of the present disclosure, a bedding capable of detecting sleeping posture is provided. The bedding may comprise: a main body portion, in which an airbag is internally provided, an outer shape of the main body portion having a front side end surface for a shoulder of a user in sleep to approach; a shoulder sensor group, comprising a plurality of shoulder sensors arranged in an array along a long side direction of the front side end surface; and a controller connected to the airbag and the shoulder sensor group, configured to determine a sleeping posture of the user according to a detection result of the shoulder sensor group, and further regulate inflation and deflation of the airbag to change a height of the main body portion, wherein, when viewed from a side along the long side direction of the front side end surface, the front side end surface has a downwardly and inwardly recessed shape (a downwardly and inwardly adducted shape), and the shoulder sensor group is positioned at a downwardly and inwardly recessed position of the front side end surface, such that the shoulder of the user during side sleeping is able to be close to the shoulder sensor group.

According to another aspect of the present disclosure, a method for detecting sleeping posture is provided, using the bedding as described above. The method may comprise: a sleeping posture determination step, wherein the controller determines the sleeping posture of the user according to the detection result of the shoulder sensor group; and a height regulating step, wherein the controller regulates the inflation and deflation of the airbag to change the height of the main body portion according to the sleeping posture of the user determined in the sleeping posture determination step.

According to yet another aspect of the present disclosure, a computer-readable storage medium is provided. The computer-readable storage medium may store executable instructions that, when executed by an information processing apparatus, cause the information processing apparatus to execute the steps of the method for detecting sleeping posture according to the other aspect of the present disclosure described above.

According to yet another aspect of the present disclosure, an apparatus capable of detecting sleeping posture is provided. The apparatus capable of detecting sleeping posture may comprise: a memory storing instructions thereon; and a processor configured to execute the instructions stored on the memory to execute the steps of the method for detecting sleeping posture according to the other aspect of the present disclosure described above.

According to a further aspect of the present disclosure, a computer program product is provided. The computer program product may comprise computer programs/instructions that, when executed by a processor, implement the steps of the method for detecting sleeping posture according to the other aspect of the present disclosure described above.

According to the technical solutions of the present disclosure, the front side end surface of the bedding is designed to have a downwardly and inwardly recessed shape, and the shoulder sensor group arranged in an array along the long side direction of the front side end surface is positioned at the downwardly and inwardly recessed position of the front side end surface, so that the shoulder of the user during side sleeping can be close to the shoulder sensor group. Thereby, the cost can be reduced while ensuring the accuracy of sleeping posture detection, and no discomfort will be caused to the human body during sleep, which can improve user comfort, help assist the smart bedding to work as a whole, and thus better improve the user's sleep quality.

### BRIEF DESCRIPTION OF THE DRAWINGS

The accompanying drawings, which form a part of the present disclosure, illustrate exemplary embodiments of the disclosure and, together with the detailed description, serve to explain the principles of the disclosure. It should be understood that the drawings are provided for purposes of illustration and not limitation.

Referring to the drawings, the present disclosure can be more clearly understood according to the following detailed description, wherein:

FIG. 1 is an exemplary schematic diagram of a bedding capable of detecting sleeping posture according to an embodiment of the present disclosure;

FIG. 2 is a schematic styling example of a front side end surface of a bedding according to an embodiment of the present disclosure;

FIG. 3 shows an exemplary schematic diagram of measurement data of shoulder sensors according to an embodiment of the present disclosure;

FIG. 4 is an exemplary schematic diagram of the configuration of shoulder sensors of a bedding capable of detecting sleeping posture according to an embodiment of the present disclosure;

FIG. 5 is an exemplary schematic diagram of the configuration of shoulder sensors of a bedding capable of detecting sleeping posture according to an embodiment of the present disclosure;

FIG. 6 is an exemplary schematic diagram of the configuration of shoulder sensors of a bedding capable of detecting sleeping posture according to an embodiment of the present disclosure;

FIG. 7 is an exemplary schematic diagram of the configuration of neck sensors of a bedding capable of detecting sleeping posture according to an embodiment of the present disclosure;

FIG. 8 is an exemplary schematic diagram of the configuration of neck sensors of a bedding capable of detecting sleeping posture according to an embodiment of the present disclosure;

FIG. 9 is an exemplary schematic diagram of the configuration of neck sensors of a bedding capable of detecting sleeping posture according to an embodiment of the present disclosure;

FIG. 10 is an exemplary overall flowchart of a method for detecting sleeping posture according to an embodiment of the present disclosure;

FIG. 11 is an exemplary flowchart of a sleeping posture determination step of the method for detecting sleeping posture according to an embodiment of the present disclosure.

Reference Numerals: 1: Main Body Portion; 2: Capacitive Sensor; 3: Piezoresistive Sensor; 4: Neck Capacitive Sensor; 4′: Neck Piezoresistive Sensor; 5: Airbag; 6: Controller; 10: Pillow.

### DETAILED DESCRIPTION

Hereinafter, preferred embodiments of the present disclosure will be described in detail with reference to the accompanying drawings. It should be noted that unless specifically stated otherwise, the relative arrangement of components, etc., numerical expressions, and numerical values set forth in these embodiments do not limit the scope of the present disclosure. Meanwhile, for ease of description, the dimensions of various parts shown in the drawings are not drawn according to actual proportional relationships. The following description of at least one exemplary embodiment is actually merely illustrative and does not serve as any limitation on the present disclosure and its application or use.

Techniques, methods, and equipment known to those of ordinary skill in the relevant art may not be discussed in detail, but where appropriate, said techniques, methods, and equipment should be considered as part of the specification.

For ease of understanding and explanation, the bedding capable of detecting sleeping posture according to the embodiments of the present disclosure is mainly described taking a pillow, mattress, etc., as examples, but this is not limiting. Those skilled in the art can understand that the technical gist of the present disclosure can be applied not only to any currently known suitable bedding but also to any future applicable bedding.

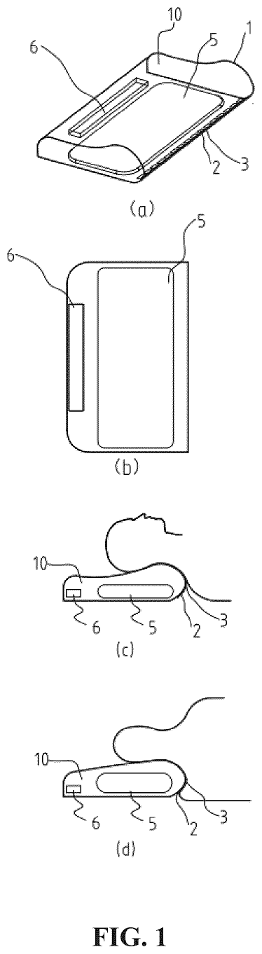

Referring now to FIG. 1, a schematic configuration example of the structure of a bedding capable of detecting sleeping posture according to an embodiment of the present disclosure will be described. In the following, for convenience, a pillow is mainly used as an example of bedding for description, but this is non-limiting. From top to bottom in FIG. 1: (a) shows a schematic perspective view of the overall structure of a pillow 10 as bedding; (b) shows a schematic top view of the pillow 10; (c) shows a schematic side view of the form of the pillow 10 when a user is lying on their back (supine); (d) shows a schematic side view of the form of the pillow 10 when a user is lying on their side.

Still referring to FIG. 1, the bedding capable of detecting sleeping posture of the embodiment of the present disclosure may comprise: a main body portion 1, in which an airbag 5 is internally provided, the outer shape of the main body portion 1 having a front side end surface for a shoulder of a user in sleep to approach; a shoulder sensor group, comprising a plurality of shoulder sensors 2 and/or 3, arranged in an array along a long side direction of the front side end surface; and a controller 6 connected to the airbag 5 and the shoulder sensor group, configured to determine the sleeping posture of the user according to a detection result of the shoulder sensor group, and further regulate the inflation and deflation of the airbag 5 to change the height of the main body portion 1, wherein, when viewed from a side along the long side direction of the front side end surface, the front side end surface has a downwardly and inwardly recessed shape, and the shoulder sensor group is positioned at a downwardly and inwardly recessed position of the front side end surface, such that the shoulder of the user during side (lateral) sleeping is able to be close to the shoulder sensor group.

More specifically, the controller 6 may include a memory and a processor coupled to the memory. The controller 6 may be connected to a barometer (not shown), which may be connected to the airbag 5 through, for example, an air pipe (not shown) to detect the air pressure of the airbag 5. The controller 6 inflates and deflates the airbag 5 via the air pipe to change the height of the pillow 10 according to the air pressure detection result from the barometer and the sleeping posture of the user determined according to the detection result of the shoulder sensor group. The barometer and the air pipe can be integrated inside the main body portion 1 without affecting detection and control.

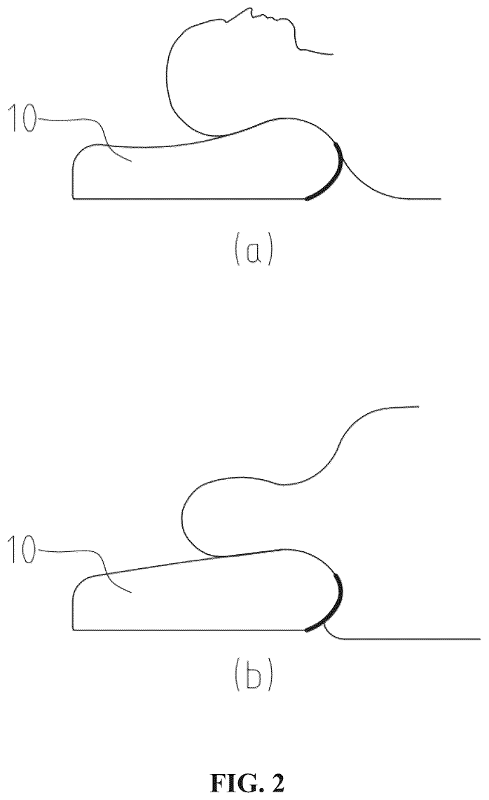

Regarding the shape of the bedding according to the embodiment of the present disclosure, as shown in FIG. 1(b), in the schematic top view of the bedding, the top surface of the main body portion 1 mainly used for supporting the user's head is shown. In the case where the bedding is a pillow 10, the long side direction on the right side of the top surface is the side approached by the shoulder of the user during sleep. On the long side direction of the right side of the top surface, a front side end surface for the shoulder of the user in sleep to approach is formed between it and the bed surface, and the long side direction of the right side of the top surface is the same as the long side direction of the front side end surface. Next, FIG. 2 shows a schematic styling example of the front side end surface of the bedding according to the embodiment of the present disclosure, wherein (a) and (b) show the situations of the user lying on the back and lying on the side, respectively. As shown by the bold line portions in FIGS. 2(a) and (b), when viewed from the side along the long side direction of the front side end surface, the front side end surface is not directly perpendicular to the bed surface, but is designed to have a downwardly and inwardly recessed shape, so that the shoulder sensor group is positioned at the downwardly and inwardly recessed position of the front side end surface, i.e., the portion shown by the bold line. As shown in FIG. 2(b), the downwardly and inwardly recessed structure of the front side end surface enables the user's shoulder during side sleeping to naturally come close to the shoulder sensor group, thereby abutting against and pressing tightly against the front side end surface.

Hereinafter, the principle of the sleeping posture detection technology of the present disclosure is briefly introduced in conjunction with the drawings. Under the action of gravity, the shapes of the parts of the human body in sleep in contact with the bedding, including the shoulder, neck, and back, depend on the different sleeping postures such as supine and side sleeping. The distribution of pressure per unit area (i.e., intensity of pressure) of the shoulder and neck on bedding such as the pillow 10 or mattress presents significantly different patterns. Among them, when lying on the side, in order for the pillow 10 to better support the side of the neck, the shoulder will naturally abut against and press tightly against the front side end surface; when the front side end surface presents an overall forward protruding shape as shown in FIG. 2, the shoulder may even squeeze into the gap between the pillow and the bed surface during sleep, i.e., the downwardly and inwardly recessed position below the front side end surface. At this time, in the shoulder sensor group arranged in an array along the long side direction of the front side end surface, several sensors located in the long side direction that are under pressure are squeezed by the shoulder with greater pressure and detect larger pressure values, while the detection pressure values of uncompressed sensors are almost zero. The spatial distribution of pressure is relatively concentrated and the pressure values are relatively large. On the other hand, when lying on the back, the shoulders only rest relatively gently against the front side end surface of the pillow, and the shoulder sensor group as a whole is squeezed by the shoulders to a lesser extent, with very small pressure values and a relatively dispersed pressure distribution. Through the novel design of the downwardly and inwardly recessed front side end surface of the bedding, combined with the cooperatively positioned shoulder sensor group, the above-mentioned laws of different pressure distributions during supine and side sleeping can be highlighted, thereby detecting the sleeping posture more accurately.

The inventors of the present application have further deeply studied the characteristics of different types of sensors and selected suitable sensor types to perform sleeping posture detection more accurately. As preferred sensor types, consideration is given to using, for example, piezoresistive sensors and capacitive sensors, which have a high cost-performance ratio. Among them, the piezoresistive sensor can directly reflect the squeezing force between the shoulder and the front side end surface. As a capacitive sensor, if a single-sheet sensor is used, as described above, it forms a capacitor between the single-sheet sensor and the human body; the smaller the distance between the sensor and the human body, the larger the capacitance. Thus, it can reflect the degree of fit between the shoulder and the front side end surface, thereby indirectly reflecting the squeezing force between the shoulder and the front side end surface.

For ease of explanation, FIG. 1 shows the case where the shoulder sensor group includes both a capacitive sensor group and a piezoresistive sensor group, but in reality, the shoulder sensor group only needs to include at least one of the capacitive sensor group and the piezoresistive sensor group. As shown in FIG. 1, the capacitive sensor group includes a plurality of capacitive sensors 2 arranged in an array along the long side direction of the front side end surface, and the piezoresistive sensor group includes a plurality of piezoresistive sensors 3 arranged in an array along the long side direction of the front side end surface. In the case of using piezoresistive sensors, in order to better collect the squeezing force between the shoulder and the front side end surface, it is preferable to configure the piezoresistive sensors close to the part where the squeezing deformation is large when the shoulder and the front side end surface interact, for example, close to the forward protruding position of the front side end surface when viewed from the side. In the case of using capacitive sensors, in order to better distinguish whether the shoulder fits with the front side end surface, it is desired to maximize the distance between the two when they tend to separate, for example, when lying on the back. In other words, it is best to configure the capacitive sensors at a part relatively far from where the shoulder and the front side end surface easily approach under various sleeping postures, that is, relatively far from the forward protruding position of the front side end surface when viewed from the side, which means configuring them at a relatively lower position. Thus, preferably, when viewed from the side along the long side direction of the front side end surface, the piezoresistive sensor group is positioned higher than the capacitive sensor group.

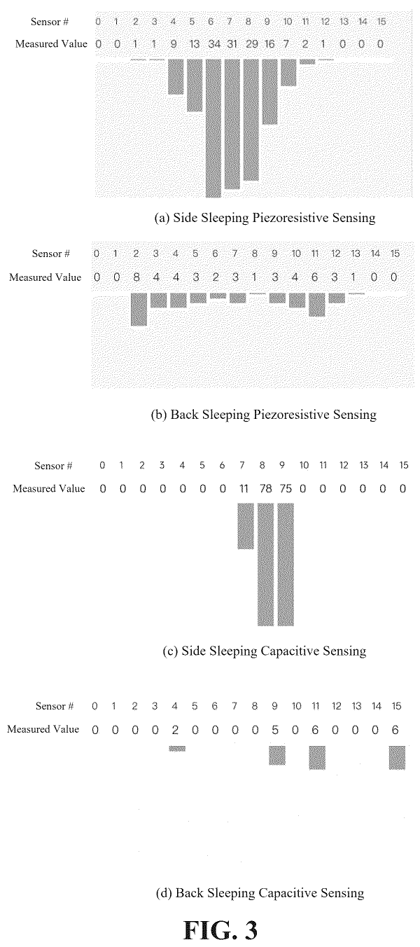

Hereinafter, examples of measurement data using the piezoresistive sensor group and the capacitive sensor group respectively will be described with reference to FIG. 3. (a) to (d) therein respectively show pressure measurement data using piezoresistive sensors during side (lateral) sleeping and back (supine) sleeping, and using capacitive sensors during side sleeping and back sleeping. In each figure, the first row of data is the sensor number, and the second row of data is the measured value; for ease of visualization, the pressure distribution is shown in the form of a histogram below each pressure measurement value. From the comparison of (a) side sleeping and (b) back sleeping in FIG. 3 using piezoresistive sensors, it can be seen that when lying on the side, since the contact area between the shoulder and the front side end surface of the pillow main body portion is significantly smaller than the contact area when lying on the back, the pressure per unit area is significantly larger, and the pressure values of the uncompressed parts drop sharply, presenting a clear spatial distribution of pressure concentration. When lying on the back, the head and neck are mainly supported by the top surface of the pillow main body portion, exerting only slight pressure on the piezoresistive sensors at the front side end portion. The shoulders on both sides, especially the acromion parts, lean relatively gently toward the front side end surface and exert similar or slightly larger pressure, presenting a pattern of small pressure amplitude and dispersed distribution overall.

On the other hand, from the comparison of (c) side sleeping and (d) back sleeping in FIG. 3 using capacitive sensors, it can be seen that when lying on the side, although the capacitive sensors are positioned lower than the piezoresistive sensors, because the shoulder abuts against and tightly fits the front side end portion, it may even squeeze into the downwardly and inwardly recessed position below the front side end surface, i.e., the gap between the pillow and the bed surface. At this time, in the narrower part where the shoulder tightly fits the front end surface side of the pillow, several capacitive sensors can detect larger capacitance as pressure values. When lying on the back, since the distance between the shoulder and the capacitive sensors is relatively far, the capacitance is very small, presenting a pattern of low amplitude and sporadic distribution overall, and there may even be cases where the overall value is 0.

Those skilled in the art can understand that the pressure measurement values in FIG. 3 are only examples used to better present the spatial distribution of pressure under various sleeping postures and do not represent a specific proportional relationship between the measurement values of piezoresistive sensors and capacitive sensors. In other words, depending on the influence of multiple variables in the actual use environment such as sensor characteristic differences, user body type and weight differences, changes in the relative position of the pillow and the human body (including left, right, up, and down), pillow height adjustment, interference from intelligent components inside the pillow, and the influence of bedding supplies such as pillowcases, the spatial distribution of piezoresistive sensor measurement values during side sleeping, for example, may also be more concentrated than the spatial distribution of capacitive sensor measurement values.

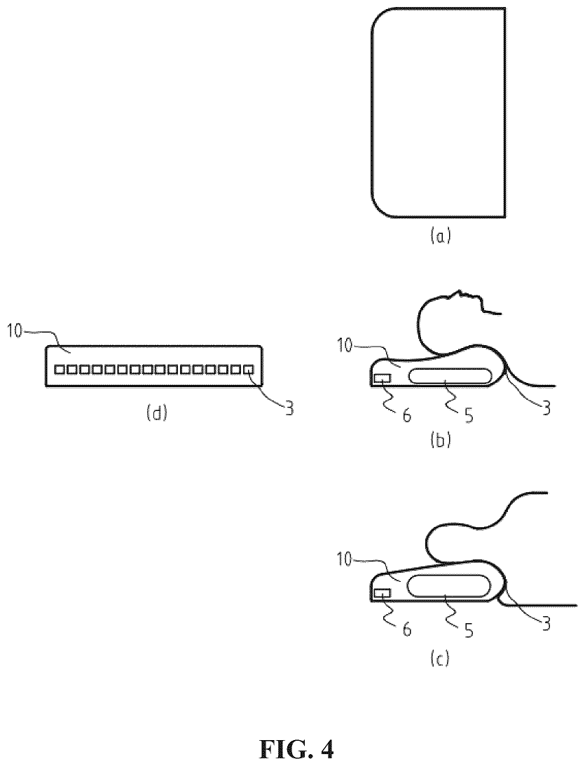

Hereinafter, the configuration of the shoulder sensors of the bedding capable of detecting sleeping posture according to the embodiments of the present disclosure will be described in detail with reference to FIGS. 4-6. Specifically, the shoulder sensor group of the pillow 10 as the bedding of the embodiment of the present disclosure includes at least one of a capacitive sensor group and a piezoresistive sensor group. The capacitive sensor group includes a plurality of capacitive sensors 2 arranged in an array along the long side direction of the front side end surface, and the piezoresistive sensor group includes a plurality of piezoresistive sensors 3 arranged in an array along the long side direction of the front side end surface. When viewed from the side along the long side direction of the front side end surface, the piezoresistive sensor group is positioned higher than the capacitive sensor group.

FIG. 4 is an exemplary schematic diagram of the configuration of the shoulder sensors of the bedding capable of detecting sleeping posture according to an embodiment of the present disclosure. (a)-(d) sequentially show a top view of the top surface of the main body portion of the pillow 10, a side view of the user lying on the back observed along the long side direction of the top surface (i.e., the long side direction of the front side end surface), a side view of the user lying on the side, and a side view of the front side end surface observed along the short side direction of the top surface. Here, as the shoulder sensor group, only a piezoresistive sensor group including a plurality of piezoresistive sensors 3 arranged in an array along the long side direction of the front side end surface is provided. For example, the piezoresistive sensor group can be positioned within a range of, for example, about 15° upward to about 30° downward from the horizontal direction.

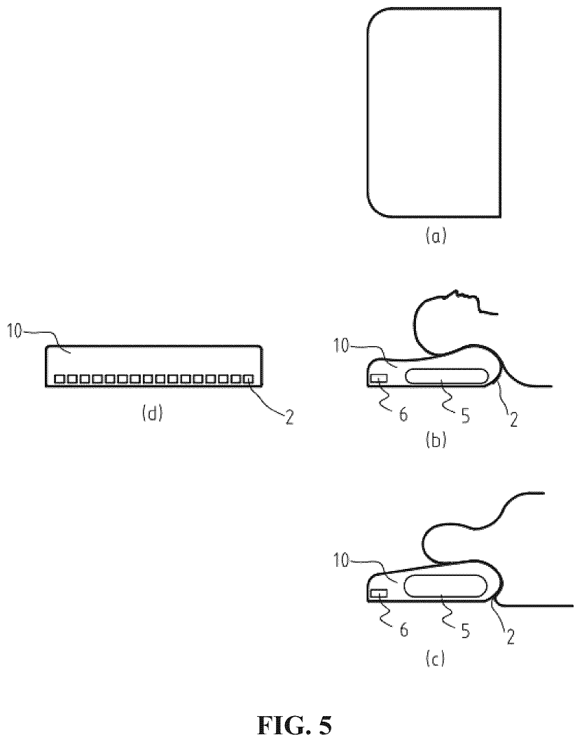

FIG. 5 is an exemplary schematic diagram of the configuration of the shoulder sensors of the bedding capable of detecting sleeping posture according to an embodiment of the present disclosure. Similar to FIG. 4, (a)-(d) sequentially show a top view of the top surface of the main body portion of the pillow 10, a side view of the user lying on the back, a side view of the user lying on the side, and a side view of the front side end surface. The difference from FIG. 4 is that, as the shoulder sensor group, a capacitive sensor group including a plurality of capacitive sensors 2 arranged in an array along the long side direction of the front side end surface is provided to replace the piezoresistive sensor group. For example, the capacitive sensor group can be positioned within a range of, for example, about 30° downward to about 90° downward from the horizontal direction. Comparing with FIG. 4, it can be seen that when viewed from the side along the long side direction of the front side end surface, the piezoresistive sensor group is positioned higher than the capacitive sensor group.

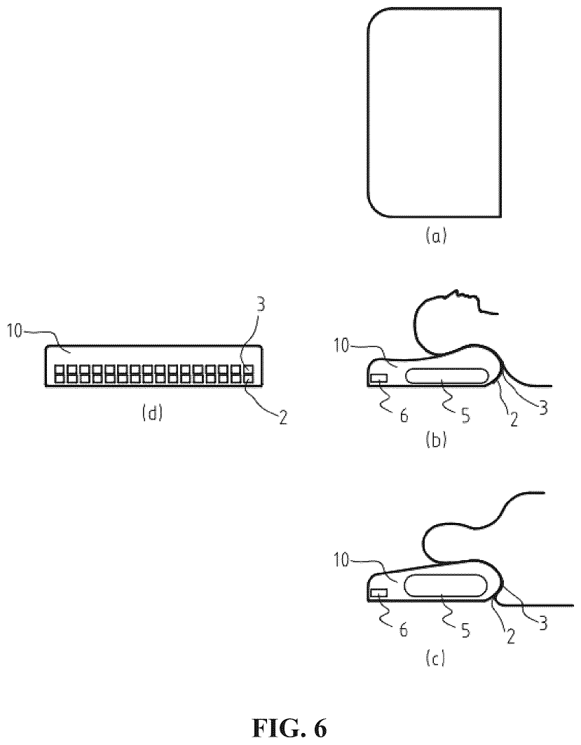

FIG. 6 is an exemplary schematic diagram of the configuration of the shoulder sensors of the bedding capable of detecting sleeping posture according to an embodiment of the present disclosure. Similar to FIGS. 4 and 5, (a)-(d) sequentially show a top view of the top surface of the main body portion of the pillow 10, a side view of the user lying on the back, a side view of the user lying on the side, and a side view of the front side end surface. The difference from FIGS. 4 and 5 is that, as the shoulder sensor group, a combination of both the capacitive sensor group and the piezoresistive sensor group is provided, wherein when viewed from the side along the long side direction of the front side end surface, the piezoresistive sensor group is positioned higher than the capacitive sensor group. At this time, in the sleeping posture detection work, the controller 6 can perform the following two-level detection: first, determine the sleeping posture of the user according to the detection result of the capacitive sensor group; if it is determined that the user is in a side sleeping position, the user's sleeping posture is finally determined as side sleeping; otherwise, the controller 6 further determines according to the detection result of the piezoresistive sensor group; if it is determined that the user is in a back sleeping position, the user's sleeping posture is finally determined as back sleeping, otherwise it is determined as side sleeping. This is because, since the capacitive sensor group is positioned relatively lower, the measurement value when lying on the back is close to 0; once the user is in a side sleeping state, the measurement value will increase significantly, so it is basically impossible to mis-determine back sleeping as side sleeping, and the accuracy when determined as side sleeping is close to 100%. However, depending on the configuration position of the capacitive sensor group, for example, when configured very low, there is a possibility of insufficient sensing even if the user is in a side sleeping state, leading to the possibility of mis-determination of side sleeping as back sleeping; in such a case, it is necessary to combine the detection of the piezoresistive sensor group for secondary determination. Through such two-level detection, the accuracy of sleeping posture detection can be improved.

Referring to the example of shoulder sensor measurement data shown in FIG. 3, the controller 6 may preset different determination thresholds for side sleeping and back sleeping as sleeping posture determination parameters, and determine the sleeping posture according to the determination thresholds. In addition, as mentioned above, due to the influence of multiple variables such as sensor characteristic differences in the actual use environment, user body type and weight differences, and changes in the relative position of the pillow and the human body, the spatial distribution of pressure measurement values under different sleeping postures will change. In this regard, the controller may include an intelligent learning module, which regulates the sleeping posture determination parameters suitable for the user according to the actual pressure distribution of the user under back sleeping and side sleeping by having the user participate in sleeping posture detection learning. For example, artificial intelligence (AI) algorithms can be used to collect data on specific users lying on their backs/sides. By learning the spatial distribution of pressure measurement values of different users under back sleeping and side sleeping, and regulating the sleeping posture determination parameters suitable for the user to train a personalized model, a more accurate real-time sleeping posture determination result for that user can be obtained.

In addition, suitable pillow heights under different sleeping postures can also be recommended according to physiological parameters including the user's height, weight, shoulder width, back thickness, cervical curvature, etc., and the firmness of the mattress. Based on the recommended pillow height, the inflation and deflation of the airbag under different sleeping postures are controlled, ultimately further improving the user's sleep quality.

In addition to the shoulder sensor group, the bedding according to the embodiment of the present disclosure may further include a neck sensor connected to the controller 6, arranged along the long side direction of the front side end surface, and positioned higher than the shoulder sensor group when viewed from the side along the long side direction of the front side end surface, such that the neck of the user in sleep is able to be close to the neck sensor.

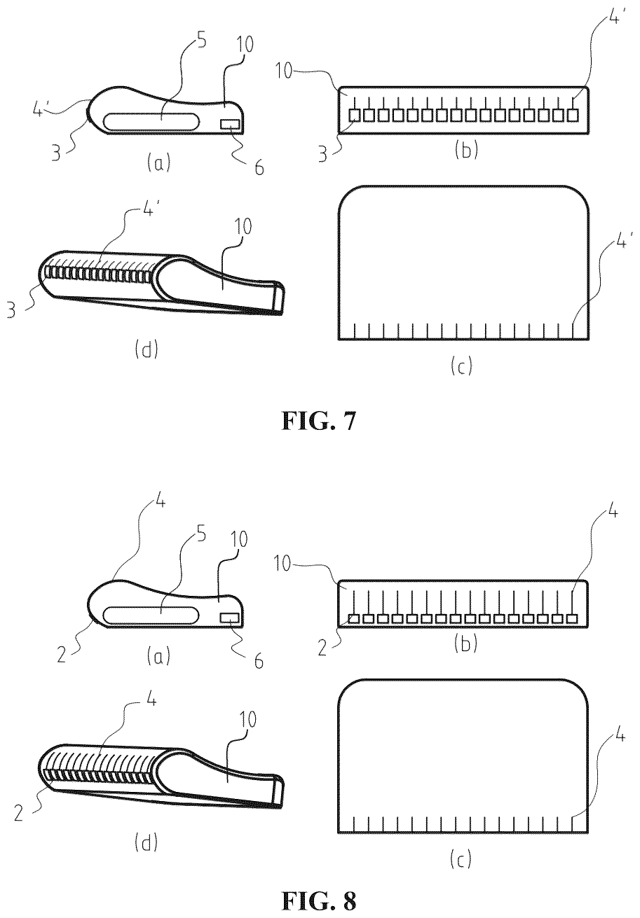

Hereinafter, the configuration of the neck sensors of the bedding capable of detecting sleeping posture according to the embodiments of the present disclosure will be described in detail with reference to FIGS. 7-9. Specifically, the neck sensor of the pillow 10 as the bedding of the embodiment of the present disclosure is generally arranged in a strip shape along the long side direction of the front side end surface. The controller 6 determines whether the user is off the pillow according to the detection result of the neck sensor. When it is determined that the user is in an off-pillow state, the controller 6 does not perform inflation and deflation control on the airbag 5.

Specifically, for example, FIG. 7 is an exemplary schematic diagram of the configuration of the neck sensors of the bedding capable of detecting sleeping posture according to an embodiment of the present disclosure, wherein (a)-(d) sequentially show a side view of the pillow 10 observed along the long side direction of the top surface, a side view of the front side end surface observed along the short side direction of the top surface, a top view of the top surface of the main body portion 1, and a perspective view of the overall structure of the pillow 10. Here, as the shoulder sensor group, only a piezoresistive sensor group including a plurality of piezoresistive sensors 3 is provided, which corresponds to the case of the configuration of the shoulder sensor group shown in FIG. 4. At this time, the neck sensor is a neck piezoresistive sensor group including respective neck piezoresistive sensors 4′ extending in a strip shape upwardly along the short side direction of the front side end surface from the respective piezoresistive sensors 3 in the piezoresistive sensor group, forming a structure in which the neck piezoresistive sensors 4′ for off-pillow detection and the piezoresistive sensors 3 in the shoulder sensor group are combined. In this case, the area of the neck piezoresistive sensors 4′ can be designed to be very small, basically not affecting the measurement of the piezoresistive sensors 3, and thus not affecting the detection of back sleeping and side sleeping. The distance between two adjacent neck piezoresistive sensors 4′ is designed to be small enough, for example, less than 6 cm, thereby ensuring that when the user is on the pillow, the neck will definitely touch the neck sensor, making the measured value (pressure) greater than 0; when the user is off the pillow, the measured value is 0. Thereby, it is possible to conveniently and accurately determine whether the user is off the pillow.

FIG. 8 is an exemplary schematic diagram of the configuration of the neck sensors of the bedding capable of detecting sleeping posture according to an embodiment of the present disclosure, wherein (a)-(d) sequentially show views similar to those in FIG. 7(a)-(d). The difference from FIG. 7 is that here, as the shoulder sensor group, only a capacitive sensor group including a plurality of capacitive sensors 2 is provided, which corresponds to the case of the configuration of the shoulder sensor group shown in FIG. 5; correspondingly, the neck sensor is a neck capacitive sensor group including respective neck capacitive sensors 4 extending in a strip shape upwardly along the short side direction of the front side end surface from the respective capacitive sensors 2 in the capacitive sensor group, forming a structure in which the neck capacitive sensors 4 for off-pillow detection and the capacitive sensors 2 in the shoulder sensor group are combined. Similar to FIG. 7, in this case, the area of the neck capacitive sensors 4 can be designed to be very small so as not to affect the measurement of the capacitive sensors 2, and thus not affect the detection of back sleeping and side sleeping. The distance between two adjacent neck capacitive sensors 4 is designed to be small enough, for example, less than 6 cm, thereby ensuring that when the user is on the pillow, the neck will definitely touch the neck sensor, making the measured value (e.g., capacitance) greater than 0; when the user is off the pillow, the measured value is 0. Thereby, it is possible to conveniently and accurately determine whether the user is off the pillow.

Those skilled in the art can understand that in FIGS. 7 and 8, for convenience, the respective neck piezoresistive sensors 4′ and neck capacitive sensors 4 extending in a strip shape upwardly along the short side direction of the front side end surface are shown as straight strips. However, the shape of the neck sensor is not limited to a straight strip, but can be various strip shapes such as curved strips, wavy strips, zigzag strips, etc., as long as the area proportion of the neck sensor in the long side direction of the front side end surface is low enough not to affect the detection of the shoulder sensor group.

In addition, although not shown, corresponding to the case of the configuration of the shoulder sensor group shown in FIG. 6, when a combination of both the capacitive sensor group and the piezoresistive sensor group is included as the shoulder sensor group, since the piezoresistive sensor group is positioned higher than the capacitive sensor group when viewed from the side along the long side direction of the front side end surface, the neck sensor at this time is, similar to FIG. 7, a neck piezoresistive sensor group including respective neck piezoresistive sensors 4′ extending in a strip shape upwardly along the short side direction of the front side end surface from the respective piezoresistive sensors 3 in the piezoresistive sensor group, forming a structure in which the neck piezoresistive sensors 4′ for off-pillow detection and the piezoresistive sensors 3 in the shoulder sensor group are combined.

By constructing the neck sensor for off-pillow detection integrally with the shoulder sensor in the above manner, the neck sensor can be manufactured at a lower cost to realize off-pillow detection.

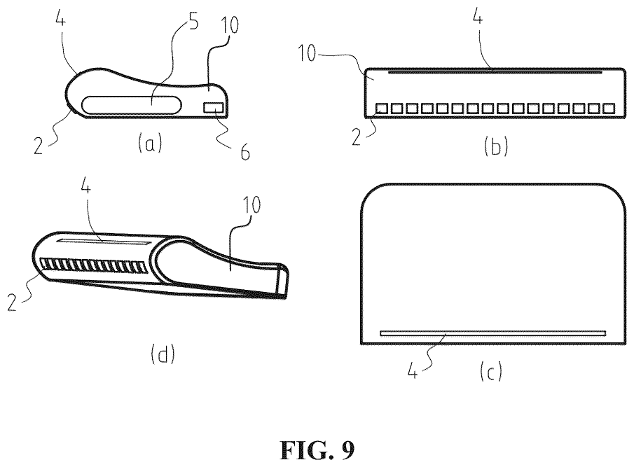

FIG. 9 is an exemplary schematic diagram of the configuration of the neck sensors of the bedding capable of detecting sleeping posture according to an embodiment of the present disclosure, wherein (a)-(d) sequentially show views similar to those in FIG. 8(a)-(d). The difference from FIG. 8 is that the neck sensor is arranged in a strip shape along the long side direction of the front side end surface. In this way, the neck sensor can be manufactured with a simpler process to realize off-pillow detection. Although not shown, the neck sensor arranged in a strip shape here can also be applied to the cases of the configuration of the shoulder sensor group in FIGS. 4 and 6. In addition, although the neck sensor is shown as the neck capacitive sensor 4 in FIG. 9 for convenience, in fact, regardless of the configuration of the shoulder sensor group, the neck sensor arranged in a strip shape can be either the neck capacitive sensor 4 or the neck piezoresistive sensor 4′.

By providing the neck sensor, the controller 6 can determine whether the user is off the pillow according to the detection result of the neck sensor. For example, when the pressure value detected by the neck sensor is 0, it can be determined that the user is in an off-pillow state; when it is determined that the user is in an off-pillow state, the controller 6 does not perform inflation and deflation control on the airbag 5. Thereby, the power consumption of the pillow 10 as bedding can be effectively reduced.

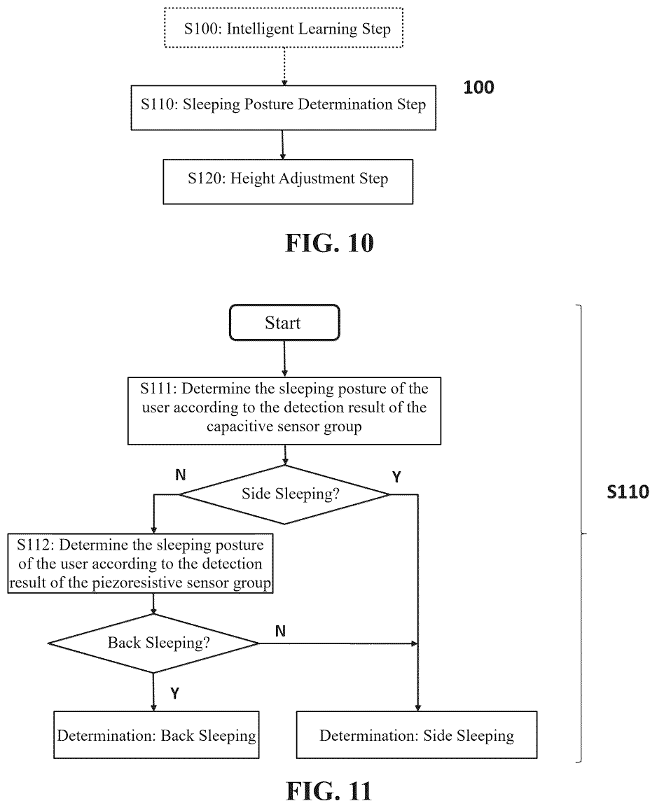

In addition, the embodiment of the present disclosure further provides a method for detecting sleeping posture. FIG. 10 is an exemplary overall flowchart of a method 100 for detecting sleeping posture according to an embodiment of the present disclosure. In this method, using, for example, the pillow 10 as bedding as described above, the method 100 includes, for example: a sleeping posture determination step S110, wherein the controller 6 determines the sleeping posture of the user according to the detection result of the shoulder sensor group; and a height adjustment step S120, wherein the controller 6 controls the inflation and deflation of the airbag 5 to adjust the height of the main body portion 1 according to the sleeping posture of the user determined in the sleeping posture determination step S110.

Wherein the method 100 may further comprise an intelligent learning step S100, in which, by involving the user to participate in sleeping posture detection learning, sleeping posture determination parameters suitable for the user are adjusted according to actual pressure distributions of the user in a back sleeping position and in a side sleeping position. Wherein, in the sleeping posture determination step S110, the method may further comprise that the controller 6 determines whether the user is off the pillow according to a detection result of the neck sensor 4 or 4′. Wherein, in the height adjustment step S120, the method may further comprise that, when it is determined in the sleeping posture determination step S110 that the user is in an off-pillow state, the controller 6 does not perform inflation or deflation control of the airbag 5. Wherein, in the height adjustment step S120, the controller 6 may perform control in such a manner that: the airbag 5 is inflated when it is detected in the sleeping posture determination step S110 that the user changes from a back sleeping state to a side sleeping state; and the airbag 5 is deflated when it is detected in the sleeping posture determination step S110 that the user changes from a side sleeping state to a back sleeping state, such that a height of the main body portion 1 when the user is in the side sleeping state is higher than a height of the main body portion 1 when the user is in the back sleeping state.

FIG. 11 shows an exemplary flowchart of the sleeping posture determination step S110 of the method 100 for detecting sleeping posture according to an embodiment of the present disclosure. When the shoulder sensor group includes both a capacitive sensor group and a piezoresistive sensor group, in the sleeping posture determination step S110, the following two-level detection can be performed: in sub-step S111, the controller 6 first determines the sleeping posture of the user according to the detection result of the capacitive sensor group; if it is determined that the user is in a side sleeping position, the user's sleeping posture is finally determined as side sleeping; otherwise, entering sub-step S112, the controller 6 further determines according to the detection result of the piezoresistive sensor group; if it is determined that the user is in a back sleeping position, the user's sleeping posture is finally determined as back sleeping, otherwise it is determined as side sleeping.

According to an embodiment of the present disclosure, a computer-readable storage medium can be provided. The computer-readable storage medium may store executable instructions that, when executed by an information processing apparatus, cause the information processing apparatus to execute the steps of the method 100 for detecting sleeping posture according to the present disclosure described above.

According to an embodiment of the present disclosure, an apparatus capable of detecting sleeping posture can be provided. The apparatus capable of detecting sleeping posture may comprise: a memory storing instructions thereon; and a processor configured to execute the instructions stored on the memory to execute the steps of the method 100 for detecting sleeping posture according to the present disclosure described above. Wherein, the memory may include, for example, a system memory, a fixed non-volatile storage medium, etc. The system memory stores, for example, an operating system, application programs, a boot loader, a database, and other programs.

According to an embodiment of the present disclosure, a computer program product can be provided. The computer program product may comprise computer programs/instructions that, when executed by a processor, implement the steps of the method 100 for detecting sleeping posture according to the present disclosure described above.

The subject matter of the present disclosure is provided as examples of apparatuses, systems, methods, and programs for implementing the features described in the present disclosure. However, in addition to the features described above, other features or variations can also be contemplated. It is contemplated that the implementation of the components and functions of the present disclosure can be accomplished with any emerging technology that may replace any of the above-described implementation technologies.

Additionally, the above description provides examples and does not limit the scope, applicability, or configuration set forth in the claims. Changes may be made to the function and arrangement of discussed elements without departing from the spirit and scope of the present disclosure. Various embodiments may omit, substitute, or add various procedures or components as appropriate. For example, features described with respect to certain embodiments may be combined in other embodiments.

Additionally, in the description of the present disclosure, although operations are depicted in a specific order in the drawings, this should not be understood as requiring that such operations be performed in the specific order shown or in sequential order, or that all illustrated operations be performed, to achieve desirable results. In certain circumstances, multitasking and parallel processing may be advantageous.

It should be understood that at least two features among the features of the present technology described herein may be combined. That is, various features described in each specific embodiment may be combined arbitrarily without distinguishing between embodiments. The embodiments disclosed this time are illustrative in all respects and not restrictive. The scope of the present disclosure is indicated not by the above description but by the claims, and is intended to include all modifications within the meaning and scope equivalent to the claims.

The case where the bedding is a pillow has been described above. The bedding capable of detecting sleeping posture according to the embodiments of the present disclosure includes not only a common single pillow. Those skilled in the art can understand that the bedding herein may also include a support for the head when the human body is in a lying position. For example, the pillow can be integrated into a mattress, and as long as a structure including the above technical gist is designed in the head support area, it also falls within the scope of the present disclosure.

The above are only used to illustrate the technical solutions of the present disclosure and are not limiting. Any other modifications or equivalent replacements made by those skilled in the art to the technical solutions of the present disclosure, as long as they do not depart from the spirit and gist of the technical solutions of the present disclosure, shall be covered within the scope of the claims of the present disclosure. The effects described in this specification are merely illustrative and not limited thereto, and there may be other effects.

1. A bedding capable of detecting a sleeping posture of a user, comprising:
a main body including an inflatable airbag, the main body defining a front side surface configured to be positioned adjacent to a shoulder of the user during sleep;
a shoulder sensor group comprising a plurality of shoulder sensors arranged in an array along a longitudinal direction of the front side surface; and
a controller operatively coupled to the airbag and the shoulder sensor group and configured to determine the sleeping posture of the user based on outputs of the shoulder sensor group and to regulate inflation and deflation of the airbag to adjust a height of the main body,
wherein, in a side view along the longitudinal direction of the front side surface, the front side surface has a downwardly recessed region, and the shoulder sensor group is disposed within the downwardly recessed region such that, during a side sleeping posture of the user, the user's shoulder is able to closely engage the shoulder sensor group.

2. The bedding of claim 1, wherein the shoulder sensor group comprises at least one of:
a capacitive sensor group including a plurality of capacitive sensors arranged in an array along the longitudinal direction of the front side surface, and
a piezoresistive sensor group including a plurality of piezoresistive sensors arranged in an array along the longitudinal direction of the front side surface,
and wherein, in the side view, the piezoresistive sensor group is positioned higher than the capacitive sensor group.

3. The bedding of claim 1, further comprising:
a neck sensor operatively coupled to the controller and arranged along the longitudinal direction of the front side surface, the neck sensor being positioned higher than the shoulder sensor group in the side view such that a neck of the user engages the neck sensor during use.

4. The bedding of claim 2, wherein, when the shoulder sensor group includes both the capacitive sensor group and the piezoresistive sensor group, the controller is configured to:
determine whether the user is in a side sleeping posture based on outputs of the capacitive sensor group as a primary determination, and, if not determined to be side sleeping, further determine whether the user is in a back sleeping posture based on outputs of the piezoresistive sensor group, such that the sleeping posture is finally determined as side sleeping or back sleeping.

5. The bedding of claim 3, wherein the neck sensor is arranged in a strip shape along the longitudinal direction of the front side surface.

6. The bedding of claim 3, wherein the controller is configured to determine whether the user is off the pillow based on outputs of the neck sensor,
and wherein, when the user is determined to be off the pillow, the controller is configured to suspend inflation and deflation control of the airbag.

7. The bedding of claim 2, further comprising:
a neck sensor operatively coupled to the controller and arranged higher than the shoulder sensor group in the side view,
wherein, when the shoulder sensor group comprises only the capacitive sensor group, the neck sensor comprises a plurality of neck capacitive sensors each extending upward from a corresponding capacitive sensor of the shoulder sensor group along a short-edge direction of the front side surface, or comprises a strip-shaped neck sensor arranged along the longitudinal direction of the front side surface,
and wherein, when the shoulder sensor group comprises the piezoresistive sensor group, the neck sensor comprises a plurality of neck piezoresistive sensors each extending upward from a corresponding piezoresistive sensor of the shoulder sensor group along the short-edge direction of the front side surface, or comprises a strip-shaped neck sensor arranged along the longitudinal direction of the front side surface.

8. The bedding of claim 1, wherein the controller comprises an intelligent learning module configured to adjust sleep posture determination parameters based on actual pressure distributions of the user under back sleep and side sleeping postures.

9. The bedding of claim 1, wherein the bedding comprises a pillow or a mattress, and wherein, when the bedding comprises a mattress, components of the bedding are integrated into a head-support region of the mattress.

10. A method for detecting a sleep posture of a user using a bedding, comprising:
determining, by a controller in the bedding, the sleeping posture of the user based on outputs of a shoulder sensor group in the bedding; and
adjusting, by the controller, a height of a main body of the bedding by regulating inflation or deflation of an inflatable airbag in the bedding based on the determined sleeping posture,
wherein the bedding comprising:
the main body, the main body including the inflatable airbag, the main body defining a front side surface configured to be positioned adjacent to a shoulder of the user during sleep;
the shoulder sensor group comprising a plurality of shoulder sensors arranged in an array along a longitudinal direction of the front side surface; and
the controller operatively coupled to the airbag and the shoulder sensor group and configured to determine the sleeping posture of the user based on outputs of the shoulder sensor group and to regulate inflation and deflation of the airbag to adjust a height of the main body,
wherein, in a side view along the longitudinal direction of the front side surface, the front side surface has a downwardly recessed region, and the shoulder sensor group is disposed within the downwardly recessed region such that, during a side sleeping posture of the user, the user's shoulder is able to closely engage the shoulder sensor group.

11. The method of claim 10, further comprising:
determining, by the controller, whether the user is off the pillow based on outputs of the neck sensor.

12. The method of claim 10, wherein, when the shoulder sensor group includes both the capacitive sensor group and the piezoresistive sensor group, the determining comprises:
performing a primary determination based on outputs of the capacitive sensor group to determine whether the user is in a side sleeping posture; and
if not determined to be side sleeping, performing a secondary determination based on outputs of the piezoresistive sensor group to classify the sleep posture as back sleeping or side sleeping.

13. The method of claim 11, wherein, when the user is determined to be off the pillow, the controller suspends inflation and deflation control of the airbag.

14. The method of claim 10, wherein adjusting the height comprises inflating the airbag when the user transitions from back sleeping to side sleeping and deflating the airbag when the user transitions from side sleeping to back sleeping, such that the height of the main body is greater during side sleeping than during back sleeping.

15. The method of claim 10, further comprising:
adjusting, by an intelligent learning module of the controller, sleep posture determination parameters based on actual pressure distributions of the user under back sleeping and side sleeping postures.

16. A non-transitory computer-readable storage medium storing instructions which, when executed by an information processing device, cause the device to perform operation steps including:
detecting a sleep posture of a user using a bedding;
determining, by a controller in the bedding, the sleeping posture of the user based on outputs of a shoulder sensor group in the bedding; and
adjusting, by the controller, a height of a main body of the bedding by regulating inflation or deflation of an inflatable airbag in the bedding based on the determined sleeping posture,
wherein the bedding comprising:
the main body, the main body including the inflatable airbag, the main body defining a front side surface configured to be positioned adjacent to a shoulder of the user during sleep;
the shoulder sensor group comprising a plurality of shoulder sensors arranged in an array along a longitudinal direction of the front side surface; and
the controller operatively coupled to the airbag and the shoulder sensor group and configured to determine the sleeping posture of the user based on outputs of the shoulder sensor group and to regulate inflation and deflation of the airbag to adjust a height of the main body,
wherein, in a side view along the longitudinal direction of the front side surface, the front side surface has a downwardly recessed region, and the shoulder sensor group is disposed within the downwardly recessed region such that, during a side sleeping posture of the user, the user's shoulder is able to closely engage the shoulder sensor group.

17-18. (canceled)
